(19) United States
(12) Patent Application Publication
(10) Pub. No.: US 2013/0103148 A1
(43) Pub. Date: Apr. 25, 2013

(54) Intraocular Lens

(71) Applicant: Bausch & Lomb Incorporated, Rochester, NY (US)

(72) Inventor: David P. Vanderbilt, Webster, NY (US)

(73) Assignee: Bausch & Lomb Incorporated, Rochester, NY (US)

(21) Appl. No.: 13/710,501

(22) Filed: Dec. 11, 2012

Related U.S. Application Data
(63) Continuation of application No. 12/917,722, filed on Nov. 2, 2010, now abandoned.

(51) Int. Cl. A61F 2/16

(52) U.S. Cl. CPC A61F 2/16
USPC 623/6.43

(57) ABSTRACT

An intraocular lens comprising: (a) monomeric units derived from at least one cationically polymerizable branched alkene monomer; (b) monomeric units derived from at least one cationically polymerizable monomer having a pendant benzocyclobutene group; and (c) a UV-absorbing benzotriazole component having the formula I.

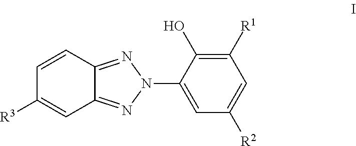

### CROSS REFERENCE

This application is a continuation of application Ser. No. 12/917,722, filed Nov. 2, 2010, which claims the benefit of Provisional Patent Application No. 61/285,571 filed Dec. 11, 2009, which is incorporated by reference herein.

### FIELD OF THE INVENTION

This invention relates to an intraocular lens.

### BACKGROUND OF THE INVENTION

The biomedical application of polyisobutylene-based materials is disclosed in U.S. Pat. Nos. 5,741,331; 6,102,939; 6,197,240; 6,545,097; and 6,855,770. The first commercial application of such materials is the use of SIBS in the TAXUS® Stent of Boston Scientific Corporation, which is regarded as the most successful launch of a biomedical device in history. SIBS is a thermoforming triblock copolymer consisting of polyisobutylene (PIB) as the rubbery center block and polystyrene (PS) as the hard side blocks. Due to the immiscibility of PIB and PS, the SIBS material has microphase-separated morphology in which PS phase forms physical crosslinks in the matrix of rubbery PIB phase. The use of a polyisobutylene-based copolymer in an intraocular lens is described in US 2009/0124773.

The human eye has an anterior chamber between the cornea and the iris, a posterior chamber behind the iris containing a crystalline lens, a vitreous chamber behind the lens containing vitreous humor, and a retina at the rear of the vitreous chamber. The crystalline lens of a normal human eye has a lens capsule attached about its periphery to the ciliary muscle of the eye by zonules and containing a crystalline lens matrix. This lens capsule has elastic optically clear anterior and posterior membrane-like walls commonly referred by ophthalmologists as anterior and posterior capsules, respectively. Between the iris and ciliary muscle is an annular crevice-like space called the ciliary sulcus.

The human eye possesses natural accommodation capability. Natural accommodation involves relaxation and constriction of the ciliary muscle by the brain to provide the eye with near and distant vision. This ciliary muscle action is automatic and shapes the natural crystalline lens to the appropriate optical configuration for focusing on the retina the light rays entering the eye from the scene being viewed.

One of the more common human eye disorders involves progressive clouding of the natural crystalline lens matrix resulting in the formation of what is referred to as a cataract. It is now common practice to cure a cataract by surgically removing the cataractous human crystalline lens and implanting an artificial intraocular lens in the eye to replace the natural lens. The prior art is replete with a vast assortment of intraocular lenses for this purpose. Intraocular lenses differ widely in their physical appearance and arrangement.

### SUMMARY OF THE INVENTION

An intraocular lens comprising: (a) monomeric units derived from at least one cationically polymerizable branched alkene monomer; (b) monomeric units derived from at least one cationically polymerizable monomer having a pendant benzocyclobutene group; and (c) a UV-absorbing benzotriazole component having the formula I

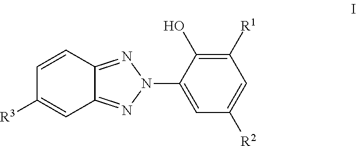

wherein R¹ and R² are independently selected from hydrogen, methyl, ethyl, propyl, iso-propyl, a C₄-C₁₀ branched alkyl with one or two optional ether linkages, C₂-C₁₆ alkylene with one or two optional ether or aromatic linkages and —NHCOR⁴, wherein R⁴ is a C₄-C₁₀ branched alkyl with one or two optional ether linkages or C₂-C₁₆ alkylene with one or two optional ether or aromatic linkages, and at least one of R¹, R² and R⁴ is a C₂-C₁₆ alkylene with one or two optional ether or aromatic linkages; and R³ is hydrogen, —OCH₃, —OC₂H₅, fluoro, chloro, bromo or iodo.

### BRIEF DESCRIPTION OF THE DRAWINGS

FIG. 1 is a front elevational view of an accommodating intraocular lens;

FIG. 2 is a side view of the FIG. 1 lens;

FIG. 3 is a detail view of a hinge of the FIG. 1 lens;

FIG. 4 is an elevational view of an accommodating intraocular lens;

FIG. 5 is a sectional view of the lens of FIG. 1 disposed in an eye, showing the lens optic in a generally anterior position and in a posteriorly vaulted position;

FIG. 6 is an elevational view of another accommodating intraocular lens;

FIG. 7 is an elevational view of another accommodating intraocular lens; and

FIG. 8 is an elevational view of another accommodating intraocular lens wherein a generally annular glare-reducing component is disposed about an optic.

### DETAILED DESCRIPTION OF THE INVENTION

An intraocular lens comprises a cured copolymer that is prepared from a cationically polymerizable, branched alkene monomer, a monomer that includes a pendent benzocyclobutene group (herein called “BCB group”), and a UV-absorbing benzotriazole component having the formula

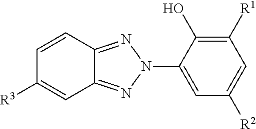

wherein R¹ and R² are independently selected from hydrogen, methyl, ethyl, propyl, iso-propyl, a C₄-C₁₀ branched alkyl with one or two optional ether linkages, C₂-C₁₆ alkylene with one or two optional ether or aromatic linkages and —NHCOR⁴, wherein R⁴ is a C₄-C₁₀ branched alkyl with one or two optional ether linkages or C₂-C₁₆ alkylene with one or two optional ether or aromatic linkages, and at least one of R¹, R² and R⁴ is a C₂-C₁₆ alkylene with one or two optional ether or aromatic linkages; and R³ is hydrogen, —OCH₃, —OC₂H₅, fluoro, chloro, bromo or iodo. The cured copolymer can further include monomeric units derived from at least one glass-forming monomer.

The branched alkene monomer can be any monomer that is both branched, contains a double bond and is cationically polymerizable. Alkenes that are not cationically polymerizable, such as 1-hexene, cannot be added to the backbone of the polymer. Similarly, many dienes, such as 1,3-butadiene, cannot propagate in a cationic polymerization reaction, as the secondary carbon in the vinyl group will frequently cause the reaction to terminate. Examples of suitable branched alkene monomers include C₄-C₁₄ branched alkenes such as isobutylene, 2-methyl-1-butene, 2-methyl-1-pentene, 2-methyl-1-hexene, and beta-pinene. Preferably, the alkene monomer is a small-chain alkene, such as a C₄-C₇ alkene. More preferably, the branched alkene monomer is an iso-olefin, such as isobutylene, 2-methyl-1-butene, or 2-methyl-1-pentene.

The cationically polymerizable branched alkene monomer preferably contains a tertiary carbon on the vinyl group in the alkene. As known by those of skill in the art, tertiary carbocations are relatively stable due to the electron-density of the surrounding carbons that stabilize the positive charge of the cation. Polyisobutylene, a preferred branched alkene monomer, as discussed above, is an example of an alkene monomer polymerizable by cationic chemical means that contains a tertiary carbon. Molecules such as propene contain secondary carbons at the vinyl group and, as known by those of skill in the art, are not cationically polymerized.

Due to the strained four-membered ring, the BCP group is converted to o-xylylene at temperatures greater than 180° C. At such elevated temperatures, the BCB group undergoes Diels-Alder reactions with dienophiles to form a six-membered ring, or reacts with itself to form an eight-membered ring. Polymers containing multiple pendant BCB groups per molecular chain can be thermally crosslinked with or without dienophiles. Each crosslink consists of a ring structure of carbon-carbon bond, which is more thermally stable than the sulfur bridge in vulcanized polymers and is stronger than the Si—O bond in silicone copolymers. The BCB crosslinking only involves heat. As long as the polymer is stable at the crosslinking temperature, there is no toxic chemical involved in order to from a cured crosslinked copolymer.

The monomer having a BCB group can be any monomer containing at least one BCB-functional moiety. It is preferred that the monomer be cationically polymerizable and be compatible with the branched alkene monomer. In one embodiment, the monomer having a BCB group has the formula

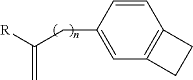

wherein R is hydrogen or an alkyl group and n is an integer selected from 0, 1, 2 or 3. Suitable monomers having a BCB group include 4-vinylbenzocyclobutene, 4-(α-alkylvinyl)benzocyclobutenes such as 2-(4-benzocyclobutenyl)-propene and 2-(4-benzocyclobutenyl)-1-butene, and 4-(2-methyl-alkenyl)benzocyclobutenes such as 2-methyl-3-(4-benzocyclobutenyl)-1-propene and 2-methyl-4-(4-benzocyclobutenyl)-1-butene. Like the branched alkene, the monomer having a pendant benzocyclobutene (BCB) group should also be cationically polymerizable. Olefins having a secondary carbon on the vinyl group are cationically polymerizable in instances when the electronegativity of the aromatic ring adjacent to the vinyl group can stabilize the carbocation. Thus, monomer olefins such as 4-vinylbenzocyclobutene can be cationically polymerized, and easily incorporated into the polymer simply by titrating it into the reaction during its polymerization. This is different than, for example, the allyl-BCB, which cannot be added to a cationic polymerization, in part because the aromatic ring in the BCB is not adjacent to the vinyl group.

BCB-type monomers having and a tertiary carbon on the vinyl group are also suitable for cationic polymerization even if the vinyl group is not adjacent to the aromatic ring of the BCB. Tertiary carbons, which become quaternary carbons during polymerization, are stabilized by the electronegativity of the surrounding carbons. Therefore, monomers having tertiary carbons on the vinyl carbons can be incorporated into a cationic polymerized reaction much in the same manner as the alkene having a tertiary carbon is incorporated. 2-Methyl-3-(4-benzocyclobutenyl)-propene is an example of this type of compound. Also preferred are monomers that draw on the electronegativity of both the surrounding carbons and the aromatic ring, for example 2-(4-benzocyclobutenyl)-propene. These type of monomers will cationically polymerize as they are stabilized both by the methyl group (as in this case of 2-(4-benzocyclobutenyl)-propene) and the aromatic ring.

As stated, the copolymer will include a UV-absorbing benzotriazole component having the formula I

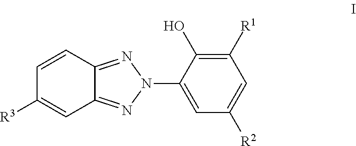

wherein R¹ and R² are independently selected from hydrogen, methyl, ethyl, propyl, iso-propyl, a C₄-C₁₀ branched alkyl with one or two optional ether linkages, C₂-C₁₆ alkylene with one or two optional ether or aromatic linkages and —NHCOR⁴, wherein R⁴ is a C₄-C₁₀ branched alkyl with one or two optional ether linkages or C₂-C₁₆ alkylene with one or two optional ether or aromatic linkages, and at least one of R¹, R² and R⁴ is a C₂-C₁₆ alkylene with one or two optional ether or aromatic linkages; and R³ is hydrogen, —OCH₃, —OC₂H₅ or fluoro, chloro, bromo or iodo.

In one embodiment of formula I, at least one of R^{I} and R² is —CH₂(CH₂)ₙC(CH₃)C═H₂, wherein n is 0, 1, 2, 3, 4, 5 and 6.

In one embodiment, the UV-absorbing benzotriazole component is of formula

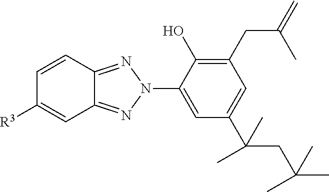

wherein R³ is defined above. This compound is referred to herein as Tin329IB, and is the UV-absorber component recited in the Examples.

In another embodiment, the UV-absorbing benzotriazole component is of formula

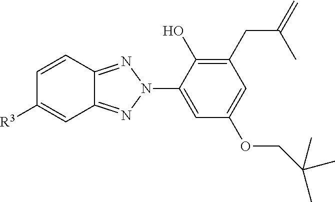

wherein R³ is defined above.

In still another embodiment, the UV-absorbing benzotriazole component is of formula

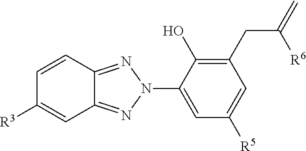

wherein R³ is defined above, and R⁵ is methyl, ethyl, propyl or iso-propyl, or a straight or branched C₁-C₈ alkoxy; R⁶ is straight or branched C₁-C₈ alkyl.

As stated the copolymer can also include monomeric units derived from a glass-forming monomer. Examples of suitable glass-forming monomers include styrenic monomers such as styrene, α-alkyl styrene (e.g., α-methyl styrene), 4-alkylstyrene, 4-alkoxystyrene, and various benzene-ring substituted styrenes. Suitable glass-forming monomers also include non-reactive glassy compounds such as norbornadiene or norbornene. The non-reactive glassy compounds are preferably bicyclic bridged systems that obey Bredt's rule, which states that the bridgeheads cannot be involved in a double bond. Compounds falling under this rule are typically inert. Preferably, the glass-forming monomer is styrene.

Methods of preparing the copolymers via cationic polymerization are well known in the art. During the carbocationic polymerization process, the BCB-monomer can be added at any time, that is, during the alkene addition, after the alkene polymerization is completed (with or without the glass-forming monomer), or both.

The number average molecular weight (Mn) of the copolymers typically range, for example, from 1000 to 2,000,000, more typically from 10,000 to 300,000, even more typically from 50,000 to 150,000, with the BCB monomeric units typically comprising 0.5 mol % to 20 mol %, even more typically 0.5 mol % to 5 mol % of the copolymer. In some embodiments, polymers have a narrow molecular weight distribution such that the ratio of weight average molecular weight to number average molecular weight (Mw/Mn) (i.e., the polydispersity index) of the polymers ranges from about 1.0 to about 2.5, or even from about 1.0 to about 1.2.

Living polymerization, i.e., a polymerization that proceeds in the practical absence of chain transfer and termination, is a desirable objective in polymer synthesis. Living cationic polymerization implies that a polymer can be grown from one or from a plurality of active sites in a controlled manner (controlled molecular weight, molecular weight distribution, end functionalities, etc.). During this growth process, different molecules can be incorporated into the backbone of the polymer, yielding polymers with well-defined structures. For example, poly(styrene-block-isobutylene-block-styrene) (“SIBS”) is a polymer where isobutylene is grown to a certain block size from two ends of a difunctional seed molecule and then styrene is infused into the reaction to cap the growing chain with glass-forming monomer segments. The result is a thermoplastic triblock polymer of polystyrene-polyisobutylene-polystyrene. One of the advantages of this type of polymer system is that depending upon the molar ratio of styrene to isobutylene, polymers can be made with durometers from Shore 20A to Shore 90D with a wide range of elongations. Another advantage of this triblock over a simple random polymerization of isobutylene and styrene is that the polymer blocks thus formed can segment into different domains which dramatically improve physical properties such as tensile strength, tear strength and compression set.

The copolymer can take various forms including linear random copolymer, linear block copolymer, star random copolymer, star block copolymer, and other hyperbranched copolymers. The copolymer composition preferably undergoes crosslinking reaction at elevated temperatures, e.g., greater than 180° C. One of ordinary skill would appreciate that the intraocular lens has improved structural characteristics and thus superior physical properties, e.g., creep resistance, heat stability under autoclaving conditions and dimensional stability.

For certain other embodiments, when the copolymers of the present invention are random copolymer, conventional polymerization can be used. Random copolymers are formed by polymerizing monomer mixtures of (a) a branched alkene monomer (e.g., isobutylene) and (b) a BCB monomer (e.g., 4-vinylbenzocyclobutene or 2-(4-benzocyclobutenyl)-propene).

In some embodiments of the present invention, block copolymers are formed by the sequential monomer addition technique using (a) a branched alkene monomer (e.g., isobutylene) and (b) a BCB monomer (e.g., 4-vinylbenzocyclobutene or 2-(4-benzocyclobutenyl)-propene). As above, in some embodiments, a mixture of BCB monomer and glass-forming monomer can be used instead of BCB monomer alone.

In one embodiment, the copolymers are block copolymers containing (a) one or more monomer blocks, which contain a plurality of units corresponding to one or more branched alkene monomer, such as isobutylene, copolymerized with (b) one or more BCB monomer, to produce a polymer comprised of polyisobutylene (or other polyalkene) dispersed with BCB crosslinkable units. In the presence of heat, for instance temperatures above 180° C., these polyisobutylene polymers containing BCB crosslinking units crosslink into 3-dimentional thermoset materials.

In another embodiment, the copolymers described above containing polyisobutylene co-polymerized with BCB can be mixed in the melt (below 180° C.) with other copolymers. The other copolymer may be, for instance, block copolymers containing (a) one or more glass-forming monomer blocks, which contain a plurality of units corresponding to one or more glass-forming monomer, such as α-methyl styrene, copolymerized with (b) one or more BCB monomer to produce a copolymer comprised of poly(α-methyl styrene) (or other glass-forming monomer) dispersed with BCB crosslinkable units. In the presence of heat, for instance temperatures above 180° C., these polyisobutylene polymers containing BCB crosslinkable units can crosslink to the poly(α-methyl styrene) polymers containing BCB crosslinkable units to form 3-dimentional thermoset materials that, depending upon the ratios of the above copolymers, can provide rubbery to stiff materials with exceptional physical and chemical properties.

In many embodiments, the polymer is formed at low temperature from a reaction mixture that comprises: (a) a solvent system appropriate for cationic polymerization, (b) one or more branched alkene monomer species, (c) an initiator, and (d) a Lewis acid coinitiator. In addition, a proton-scavenger is also typically provided to ensure the practical absence of protic impurities, such as water, which can lead to polymeric contaminants in the final product. An inert nitrogen or argon atmosphere is generally required for the polymerization.

Polymerization can be conducted, for example, within a temperature range of from room temperature to −100° C., more typically from about 0° C. to −70° C. Polymerization times are typically those times that are sufficient to reach the desired conversion.

Among the solvent systems appropriate for cationic polymerization, many of which are well known in the art, include: (a) C₁-C₄ halogenated hydrocarbons, such as methyl chloride and methylene dichloride, (b) C₅-C₈ aliphatic hydrocarbons, such as pentane, hexane, and heptane, (c) C₅-C₁₀ cyclic hydrocarbons, such as cyclohexane and methyl cyclohexane, and (d) mixtures thereof. For example, in some embodiments, the solvent system contains a mixture of a polar solvent, such as methyl chloride, methylene chloride and the like, and a nonpolar solvent, such as hexane, cyclohexane or methylcyclohexane and the like.

Initiators for living carbocationic polymerization are commonly organic ethers, organic esters, organic alcohols, or organic halides, including tert-ester, tert-ether, tert-hydroxyl and tert-halogen containing compounds. Specific examples include alkyl cumyl ethers, cumyl halides, alkyl cumyl esters, cumyl hydroxyl compounds and hindered versions of the same, for instance, dicumyl chloride, 5-tert-butyl, 1,3-dicumyl chloride, and 5-tert-butyl-1,3-bis(1-methoxy-1-methylethyl)benzene. 5-tert-butyl-1,3-bis(1-methoxy-1-methylethyl)benzene is the preferred initiator for cationic polymerization and may be prepared through the methods disclosed in Wang B. et al., “Living carbocationic polymerization XII. Telechelic polyisobutylenes by a sterically hindered bifunctional initiator” Polym. Bull. (1987)17:205-11; or Mishra M. K., et al., “Living carbocationic polymerization VIII. Telechelic polyisobutylenes by the MeO(CH₂)₂C-p-C₅H₄—C(CH₃)₂ OMe/BCl₃ initiating system” Polym. Bull. (1987)17:7-13. The initiators used for crosslinkable polyolefins described in this invention include mono or multifunctional initiators.

The polymeric material as described herein provides an index of refraction in the range between 1.48 and 1.56 as compared to an index of refraction between 1.25 and 1.42 for silicone rubber. The higher index of refraction provides greater magnification as compared to silicone rubber, which enables the IOL realized from the polymeric material described herein to be thinner than silicone rubber IOLs. A thin IOL is advantageous as it can be introduced into lens capsule through a smaller size cannula and thus reduces the size of the surgical incision into the lens capsule. The reduced size incision minimizes the chance of a surgically-induced astigmatism. Moreover, it is contemplated that the thin IOL can be inserted into a lens capsule through an incision of less than 2.5 mm, particularly less than 2.0 mm.

When PIB is used as part of the polymeric material of the IOL, the PIB is typically synthesized in organic solvent using a Lewis acid as an initiator. One such Lewis acid that is preferred for this application is titanium tetrachloride. In order to quench the reaction, chemicals such as alcohols (methanol, for example) are added in excess to the reaction stoicheometry which immediately quenches the reaction by neutralizing titanium tetrachloride. At completion of the reaction, titanium tetrachloride is converted into various salts of titanium, including titanium dioxide, titanium methoxide, and the like. In addition, depending upon the reaction vessel used, various salts of titanium can form with materials inherent to the reaction vessel, especially if the vessel is comprised of stainless steel—these salts render the material black with time. Nevertheless, a consequence of adding these reactant materials is that in order to render the material clean and highly transparent, these excess materials and their byproducts must be removed from the polymer upon completion of the reaction. These remnant salts and other unwanted chemicals are preferably washed from the PIB material by washing the polymer in a separatory funnel with salt water, with pure water and with repeated precipitations in excess polar solvent (such as isopropanol, acetone, methanol, ethanol and the like). Other well-known washing procedures can also be used. Note that if the material is not washed of salts, these hygroscopic salts begin to draw in water when the material is equilibrated in water. Voids where the salts have been trapped are readily viewed under scanning electron microscopy and these voids become filled with water as the salt is dissolved out. As water has a refractive index of approximately 1.33 and material has a refractive index of 1.53, the difference in refractive index is sufficient to render the polymer cloudy and at times totally opaque. If the material is washed appropriately, these salts are removed and voids no longer exist.

The UV-absorbing benzotriazole component can be copolymerized with the alkene monomer and the BCB monomer via a cationic polymerization. Alternatively, the UV-absorbing benzotriazole component can be blended with the copolymer in the melt phase of forming an intraocular lens and once the copolymer is crosslinked at the higher (curing) temperature, the UV-absorbing benzotriazole component is locked into the polymer.

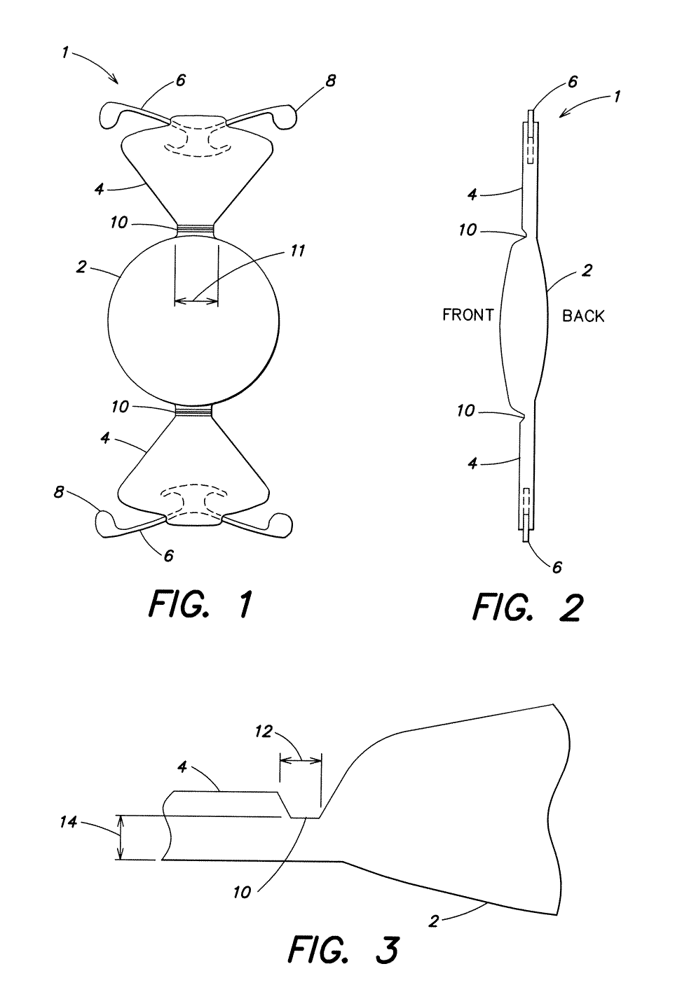

Turning now to the Figures, one embodiment of an accommodating intraocular lens is illustrated in FIGS. 1 to 3. An intraocular lens 1 formed as a flexible solid optic 2 made of the described copolymer, and flexible extending haptics 4 of any suitable form but preferably triangular plate haptics which are capable of multiple flexations without damage, and preferably, also formed of the copolymer. The optic 2 and haptics 4 preferably are uniplanar, and as shown two haptics 4 extend distally from opposite sides of the optic 2. Fixation and centration fingers 6 are provided at the distal ends of the haptics 4.

A typical length for the lens 1 is 10.5-11.5 mm, and the optic 2 typically is a 4.5 mm to 6.0 mm diameter optic. The fingers 6 preferably are approximately 5.0 mm wide and comprise four-point fixation loops that extend distally when the lens is put into any insertion cartridge. The ends 8 have a slightly different configuration and aid in indicating to the surgeon that the lens is right side up with the hinges in a proper position.

As shown, the haptics 4 have a triangular shape, with a narrow cross-section adjacent to the optic and wider at the outer ends. A hinge 10 is provided between the haptics 4 and the outer periphery of the optic 2, and it is particularly desirable to have a wide elastic base 10 to the hinge to allow the optic 2 to move forward more by stretching of the thin hinge base with the increase in vitreous cavity pressure, which can potentially provide more anterior movement of the lens. A typical hinge width 11 is 0.4 mm to 4.0 mm, and preferably with a hinge base width longitudinally as indicated by arrow 12 of 0.1 mm to 0.8 mm and preferably 0.5 mm, and a thickness range as indicated by arrow 14 of 0.06 mm to 0.4 mm, and preferably 0.12 mm, as indicated in FIG. 3. The wider hinge base stretches somewhat like an elastic band to facilitate greater anterior movement of the optic 2.

The hinges 10 are preferably on the anterior side as shown, and the round end 8 of loops 6 on the right as seen in FIG. 1 indicates that the hinge is uppermost. End 8 is round. The wider loops 6 minimize the anterior vault of the lens for distance vision and therefore provide better distance vision.

There can be a sharp edge around the posterior surface of the optic 2. To reduce the migration of cells across the posterior capsule of the lens post-operatively and thereby reduce the incidence of posterior capsular opacification and the necessity of YAG posterior capsulotomy.

A second embodiment of a described intraocular lens comprises haptics extending in a longitudinal direction between opposite portions of the equator of a capsular bag of an eye. The lens comprises an asymmetrical optic of substantially greater dimension transversely of said longitudinal direction and of lesser dimension in said longitudinal direction. The haptics extend oppositely longitudinally from the optic to engage the equator of the capsular bag. The haptics extend from capsular bag equator portions to attachment at opposite portions of the optic, whereby increased posterior vaulting of the optic is provided by the elongated haptics. The lens can also include an optic with a linear edge portions at longitudinally opposite sides with haptics being hinged to said opposite linear portions. The linear edge portions are indented from the periphery of the optic to enable elongation of the haptics. The lens may further include a light-transmitting skirt disposed about at least a portion of the periphery of the optic for reduction of glare impinging upon the retina of the eye.

Preferably, the optic has a longitudinal dimension of about 4.5 mm and a transverse dimension of about 6.0 mm. Also, the haptics can have transversely extending peripheral loop portions for engagement in the capsular bag equator.

Preferably, the haptics are hingedly mounted to the optic by flexible portions thereof adjacent to the optic. In one example, the haptics are hinged to the optic by grooved hinged portions of the haptics adjacent the optic.

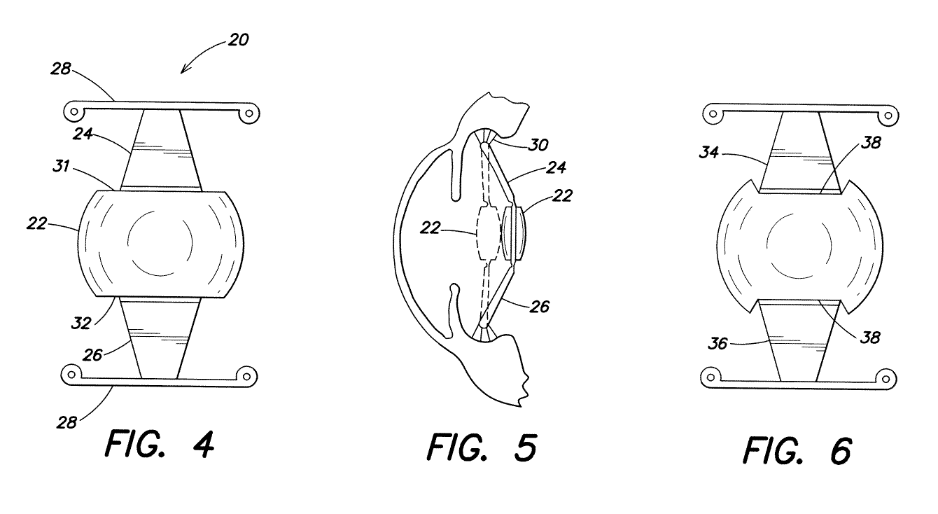

As illustrated in FIGS. 4 and 5, an accommodating lens 20 is shown as comprising an optic 22 and haptics 24, 26 extending oppositely therefrom and having loops 28 extending transversely thereof for engagement in the equator or rim of a capsular bag of an eye.

As shown, the lens is shortened in the longitudinal direction of haptics 24, 26 extension and elongated in the transverse direction, and the haptics are elongated in the longitudinal direction. From the geometry of the features and components, including the ciliary muscle 30, the haptics and the optic, it will be understood that the elongated haptics provide increased posterior vaulting of the optic, as indicated in FIG. 5. The optic thus has a somewhat oval configuration, with flat straight portions 31,32 hinged to the haptics. The lens provides improved, enhanced accommodation by increased posterior vaulting of the optic, while maintaining a maximal optical zone for accurate vision.

The optic 22, while relatively wide and enlarged in the direction transverse to the longitudinal direction of the haptics, and relatively short in the longitudinal direction, nevertheless has a full optical zone to provide full optical effect transmitted to the retina of the eye. Whereas artificial intraocular lenses typically have optical zones of less than 5.0 mm in diameter, particularly lenses with haptics staked into optics, the present invention provides optical zones of about 6.0 mm transversely and about 4.5 mm longitudinally.

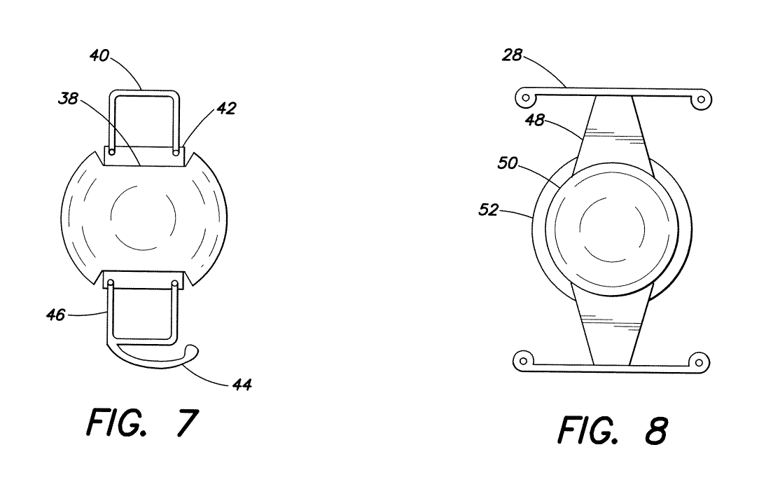

FIGS. 6 and 7 show embodiments of the invention wherein generally circular optics have indented linear portions 38, to which haptics 34, 36 are hingedly connected. FIG. 7 shows a lens with indentations 38 at which are hingedly mounted haptics of generally rectilinear rod-like configuration, the haptics having plate elements 42 hingedly mounted to the optic. A loop haptic portion 44 extending transversely from an outer edge portion of a haptic 46 to aid in centering the lens within the capsular bag of the natural human lens. A haptic 40 is mounted on the other side of the lens.

FIG. 8 shows an embodiment wherein haptics 48 are hingedly mounted relative to an optic, and disposed about an optic 50 is a thin, annular transparent or translucent light-transmitting member 52 which reduces edge glare imposed on the retina.

As is well known in the art, an intraocular lens as described is implanted in the capsular bag of the eye after removal of the natural lens. The lens is inserted into the capsular bag by a generally circular opening cut in the anterior capsular bag of the human lens and through a small opening in the cornea or sclera. The outer ends of the haptics, or loops, are positioned in the cul-de-sac of the capsular bag. The outer ends of the haptics, or the loops, are in close proximity with the bag cul-de-sac, and in the case of any form of loops, such as, the loops are deflected from the configuration. The ends or knobs of the loops are provided on the outer end portions of the loops for improved securement in the capsular bag or cul-de-sac by engagement with fibrosis, which develops in the capsular bag following the surgical removal of the central portion of the anterior capsular bag.

The inner ends of the loops may be either integrally formed from the same material as the haptics or the loops may be of a separate material such as polyimide. The loops if formed of a separate material are molded into the terminal portions of the haptics such that the flexible material of the loop can extend by elasticity along the internal fixation member of the loop.

### EXAMPLE 1

### Synthesis of poly(st-co-4VBCB)-polyIB-co-Tin329IB)poly(st-co-4VBCB) Triblock Copolymer

337 mL of methylcyclohexane (MeCHx) and 0.15 g of Tin329IB are added to a glass reaction vessel equipped with mechanical stirrer, stainless steel serpentine tubing for liquid nitrogen cooling, stainless steel tubing for feeding methyl chloride (MeCl) and isobutylene (IB) into the vessel, a thermocouple with temperature controller, a nitrogen bubbler and an addition dropping funnel. The reaction mixture is cooled down to −80° C. with liquid nitrogen. Sequentially MeCl (189 g) and IB (20 g) are added through the feeding line immersed in the liquid, so that the gases condense into the liquid. While gases are being condensed, an initiator solution (HDCE/DTBP/MeCHx 0.28 g/0.45 mL/15 mL) is added through a port on the reactor lid. TiCl₄ (3.5 mL) is added with a 10 mL glass syringe through an opening in the reactor lid. The addition of the TiCl4 starts the IB cationic polymerization (the timer is started). Forty minutes later, about 0.4 mL of reaction mixture is withdrawn from the reactor and quenched in excess methanol. A mixture of styrene/4VBCB/MeCHx (7 mL/2 mL/20 mL) is added slowly into the reactor through a syringe over 2 minutes. After 6 minutes, the reaction is quenched with excess methanol.

The reaction mixture is stored under the fume hood overnight. The top layer is separated and washed repeatedly with distilled water until neutral. The solution is precipitated into isopropyl alcohol, followed by dissolution in toluene and precipitation in isopropyl alcohol again. The precipitate is dried in a vacuum oven at 60° C. until constant weight.

### EXAMPLE 2

### Synthesis of poly(IB-co-4VBCB-coTin329IB) Random Copolymer

337 mL of methylcyclohexane (MeCHx) is added to a glass reaction vessel equipped with mechanical stirrer, stainless steel tubing for liquid nitrogen cooling, stainless steel tubing for feeding methyl chloride (MeCl) and isobutylene (IB) into the vessel, a thermocouple with temperature controller, a nitrogen bubbler and an addition dropping funnel. The methylcyclohexane is cooled down to −80° C. with liquid nitrogen. Sequentially MeCl (189 g) and IB (5 g) are added through the feeding line immersed in the methylcyclohexane, so that the gases condense into the liquid. While gases are being condensed, an initiator solution (HDCE/DTBP/MeCHx 0.28 g/0.45 mL/15 mL) is added through a port on the reactor lid. TiCl₄ (3.5 mL) is added with a 10 mL glass syringe through an opening in the reactor lid, which starts the IB polymerization (the timer was started). Five minutes later, 36 g of IB, and a mixture of 1.5 g 4VBCB and 0.2 g of Tin329IB are added slowly, taking 7 min, and 9.5 min, respectively. After all 4-VBCB/Tin329IB is added, the reaction is kept going for 60 min. Reaction aliquots are removed at 5, 30 and 60 min. The reaction is quenched with excess methanol.

The reaction mixture is stored under the fume hood overnight. The top layer is separated and washed repeatedly with distilled water until neutral. The solution is precipitated into isopropyl alcohol, followed by dissolution in hexane and precipitation in isopropyl alcohol again. The precipitate is dried in a vacuum oven at 60° C. until constant weight.

The polymer is characterized by GPC and ¹H-NMR. GPC RI traces of the reaction aliquots show that the molecular weight increases with reaction time. Little change is observed in molecular weight (about 10%) from 30 min to 60 min, as the polymerization approaches completion. The incorporation of 4-VBCB into the copolymer is further confirmed by ¹H-NMR spectroscopy. A singlet at about 3.1 ppm can be observed in the ¹H-NMR spectrum, which is attributed to the strained ring of BCB. When heated up to 240° C. for 10 min, the copolymer becomes thermally crosslinked.

### EXAMPLE 3

### Thermal Crosslinking of poly(st-co-4VBCB)-polyIB-coTin329IB)-poly(st-co-4VBCB) Triblock Copolymer

A sample (0.2 g) of the poly(st-co-4VBCB)-polyIB-coTin329IB -poly(st-co-4VBCB) triblock copolymer as formed above is placed between two Teflon films, and the films placed between two flat metal plates. The resulting structure is placed in a hot press (250° C.) for 10 minutes with virtually no pressure applied. The Teflon films are removed from the plates and cooled to room temperature. A small piece of the heat treated polymer is placed in a vial containing THF. The film remains insoluble overnight and longer, indicating that the polymer film is crosslinked.

Polymer samples were thermally treated at different temperatures for 10 minutes. At temperatures above 220° C., the resultant polymer is insoluble in THF. At a temperature of 200° C., the resultant polymer is soluble in THF. For thermal crosslinking below 220° C., extended time period (>10 min) may be necessary.

We claim:

1. An intraocular lens comprising: (a) monomeric units derived from at least one cationically polymerizable branched alkene monomer;(b) monomeric units derived from at least one cationically polymerizable monomer having a pendant benzocyclobutene group; and (c) a UV-absorbing benzotriazole component having the formula I 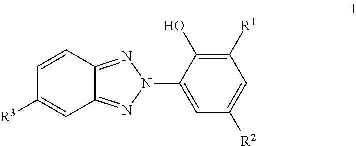
wherein R¹ and R² are independently selected from hydrogen, methyl, ethyl, propyl, iso-propyl, a C₄-C₁₀ branched alkyl with one or two optional ether linkages, C₂-C₁₆ alkylene with one or two optional ether or aromatic linkages and —NHCOR⁴, wherein R⁴ is a C₄-C₁₀ branched alkyl with one or two optional ether linkages or C₂-C₁₆ alkylene with one or two optional ether or aromatic linkages, and at least one of R¹, R² and R⁴ is a C₂-C₁₆ alkylene with one or two optional ether or aromatic linkages; and R³ is hydrogen, —OCH₃, —OC₂H₅, fluoro, chloro, bromo or iodo.

2. The intraocular lens according to claim 1, further comprising monomeric units derived from at least one glass-forming monomer.

3. The intraocular lens according to claim 1, wherein the at least one branched alkene monomer is an isoolefin.

4. The intraocular lens according to claim 3, wherein the isoolefin is isobutylene.

5. The intraocular lens according to claim 1, wherein the monomer having a pendant benzocyclobutene group has the formula 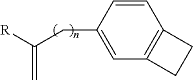
R is hydrogen or an alkyl group and n is an integer selected from 0, 1, 2 or 3.

6. The intraocular lens according to claim 1, wherein the monomer having a pendant benzocyclobutene group is selected from the group consisting of 4-vinylbenzocyclobutene, 2-(4-benzocyclobutenyl)-propene, and 2-methyl-3-(4-benzocyclobutenyl)-propene.

7. The intraocular lens according to claim 2, wherein the glass-forming monomer is present and selected from the group consisting of styrene, indene, α-methylstyrene, p-tert-butylstyrene, p-chlorostyrene, p-methoxystyrene, p-tert-butoxystyrene, p-hydroxystyrene, norbornene, and any one mixture thereof.

8. The intraocular lens according to claim 1, wherein the alkene monomer is isobutylene, the monomer having a pendant benzocyclobutene group is 4-vinylbenzocyclobutene, and the glass-forming monomer is present and is styrene.

9. The intraocular lens according to claim 1, wherein the UV-absorbing benzotriazole component has at least one of R¹, R² and R⁴ as a C₆-C₁₀ branched alkyl.

10. The intraocular lens according to claim 1, wherein the UV-absorbing benzotriazole component is of formula

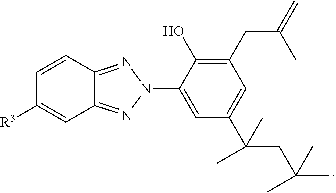

11. The intraocular lens according to claim 1, wherein the UV-absorbing benzotriazole component is of formula

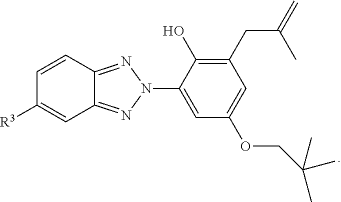

12. The intraocular lens according to claim 1, wherein the UV-absorbing benzotriazole component is of formula 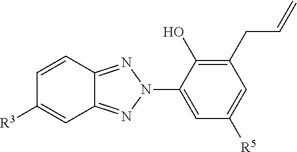
wherein R⁵ is methyl, ethyl, propyl or iso-propyl.

13. The intraocular lens according to claim 1, wherein the lens has an index of refraction from 1.52 to 1.55.

14. The intraocular lens according claim 1, further comprising hinges between the haptics and the outer periphery of the optic to allow the optic to move forward with the increase in vitreous cavity pressure.

15. The intraocular lens according to claim 14, wherein the hinges have a hinge width of 0.4 mm to 6.0 mm, a hinge base width of 0.1 mm to 0.8 mm, and a hinge thickness of 0.06 mm to 0.4 mm.

16. The intraocular lens according to claim 14, wherein the optic is an asymmetrical optic of substantially greater dimension transversely of the longitudinal direction and of lesser dimension in the longitudinal direction.
